(19) United States
(12) Patent Application Publication
(10) Pub. No.: US 2010/0184733 A1
(43) Pub. Date: Jul. 22, 2010

(54) SKIN TREATMENT COMPOSITION

(75) Inventors: Cornelis Gerrit Nijs Korevaar, Oegstgeest (NL); Mike Farwick, Essen (DE); Juergen Meyer, Essen (DE); Peter Lersch, Dinslaken (DE); Wilma Reidick, Bottrop (DE); Christian Weitemeyer, Essen (DE)

Correspondence Address: SCULLY SCOTT MURPHY & PRESSER, PC, 400 GARDEN CITY PLAZA, SUITE 300, GARDEN CITY, NY, 11530, US

(73) Assignee: EVONIK GOLDSCHMIDT GMBH, Essen (DE)

(21) Appl. No.: 12/445,365

(22) PCT Filed: Oct. 13, 2006
(86) PCT No.: PCT/EP2006/009919

§ 371 Date: Mar. 19, 2010

(51) Int. Cl. A61K 31/575; A61P 17/02

(52) U.S. Cl. 514/171

(57) ABSTRACT

The invention discloses skin treatment compositions consisting essentially of a mixture of ceramides, cholesterol, free fatty acids—the mixture being able to mimic skin identical lamellar structures—and nonionic emulsifiers. These compositions are liquid and pumpable at room temperature which means that they can be incorporated easily into cosmetic formulations with no need for heating. Furthermore, the skin treatment compositions allow the stable inclusion of long-chain ceramides into oil-in-water emulsions as well as into water-in-oil emulsions. Inclusion of short chain ceramides that can act as dermatological regulators did not affect the lamellar lipid organization. The skin treatment compositions are claimed as well as their use in cosmetic and dermatological formulations.

### FIELD OF THE INVENTION

This invention relates to topical compositions consisting of lipids incorporated into lamellar structures preventing crystallisation for optimised bioavailability and application to human skin.

### BACKGROUND AND PRIOR ART

Skin is a highly complex tissue acting as a protector against physical, chemical and biological attacks. It plays a crucial role in the protection against dehydration and the control of body temperature (A Short Textbook of Cosmetology, K F de Polo (Ed.), Verlag für chemische Industrie, H. Ziokowski GmbH, Augsburg (D), 1998). This barrier is provided by the “horny layer” (stratum corneum, SC), representing the outermost layer of the epidermis. The horny layer is a thin inert, water-retaining barrier which both regulates the moisture content of the skin and protects it against external influences.

Due to its structure it is often compared to a brick wall in which the nonviable corneocytes are embedded like bricks in a matrix of lipids (“Mortar”) (Elias P M, J. Invest. Dermatol. 80 (Suppl 1), 44 (1983)). The lipid mixture is assembled into densely packed lamellar structures consisting of ceramides, cholesterol and fatty acids. In the literature different compositions of skin lipids are given depending on extraction and analytical methods exerted and the origin of the skin used for analysis. On a weight basis, these lipids constitute of approximately 47% by weight ceramides, 24% by weight cholesterol, 11% by weight free fatty acids and 18% by weight cholesterol esters (Rawlings A V, Int. J. Cosmet. Sci. 25, 1-33 (2003)).

The lipid environment of the stratum corneum is an essential factor for maintaining the skin's equilibrium. As a result of age, health or environmental conditions, changes in the lipid composition occur, leading to a weakening of the barrier function (Rawlings, A. V. et al., J. Invest. Dermatol. 103, 731-740 (1994); Motta S; Arch Dermatol. 1994; 130, 452-456; Choi et al, J Invest Dermatol. 2005 Mar., 124(3), 587-595). These findings lead to the concept that ceramides in combination with cholesterol and fatty acids are valuable components of skin care products, since the topical application of such products can replenish low levels of stratum corneum lipids.

It is well known in the literature that the topical application of lipid mixtures containing ceramides, fatty acids and cholesterol improves the skin performance under various suboptimal conditions, e.g. barrier recovery in chronologically aged skin (Zettersten et. al., J Am Acad Dermatol. 1997 Sep., 37, 403-408), in psychologically stressed skin (Choi et al, J Invest Dermatol. 2005 March, 124(3), 587-595) or improving the barrier properties and the clinical condition of the skin in contact dermatitis (Beradesca et. al., Contact Dermatitis, 2001, 45, 280-285).

EP 0 644 764 (Elias et. al.) describes specific lipid mixtures consisting of acylceramides, ceramides or glucosylceramides together with cholesterol and/or fatty acids for epidermal moisturization and repair of barrier function. The invention is related to the treatment of skin diseases which display hyperproliferation and disruptions of the barrier function.

The activity of short chain ceramides on keratinocyte proliferation and differentiation has been described in the literature (Pillai et al. J Invest Dermatol. Symp. Proceed. 1, 39-43). U.S. Pat. No. 5,578,641 (Jackson et. al.) described the topical applications of one or more ceramide pathway intermediates or precursors for the supplementation of skins own ceramide production pathways in the epidermis. Listed structures include free sphingoid bases and their N-acyl derived ceramides with a carbon chain length of 1 to 10 atoms. The invention is related to the treatment of dry and/or (photo-)damaged skin for reducing or delaying wrinkle formation.

EP 0 975 325 (Lambers) describes compositions comprising a combination of a free sphingoid base and a ceramide showing a positive synergistic effect on lipid barrier function.

The physical organization of the membrane bilayer structure is crucial for an effective skin barrier and this is provided by a lipid lamellar assembly in a tightly packed or thorhombic configuration. Detailed research on the assembly of SC lipids has been performed establishing the so-called “Sandwich model” (Bouwstra J et al., Skin Pharmacol. Appl. Skin Physiol. 14, 52-62 (2001)). By electron microscopy and x-ray diffraction the SC lipids are observed as alternating broad/narrow/broad sequences of bilayers representing two broad lipid layers with crystalline structure separated by a narrow central lipid layer with fluid domain. The optimal ratio for the topical application of the stratum corneum lipids ceramides, cholesterol and fatty acids is in the range of 1:1:1 on a molar basis. The structural organisation of the stratum corneum lipids in vivo can be mimicked by lipid mixtures prepared with well-defined synthetic ceramides 1, 2, 3 and 9 (de Jager et al, J. Lipid Res. 2005 Dec., 46(12), 2649-2656). Using the more recent lettering system of Ceramides classification (Motta et al. BBA, 1993, 1182, 147-151) these Ceramides are called Cer(amide) EOS, Cer NS, Cer NP and Cer EOP respectively.

Although the dermatological importance of ceramides is known, it still remains a challenging target to incorporate ceramides in a stable way in cosmetic formulations. Stable in that respect means that not only the formulation is physically stable but also that ceramides do not recrystallize out of these formulations.

There have been publications explaining how ceramides can be included in cosmetic formulations avoiding this crystallization phenomenon (e.g. T. Dietz, P. Hameyer, SOFW-Journal (5), 2-9 (2003)). In these studies it was shown on the example of Ceramide 3 (NS) that up to 1% by weight of this ceramide can be included into cosmetic oil-in-water emulsions without crystallization. As a crucial step in successfully formulating ceramides it was pointed out that the ceramides had to be dissolved in the oil phase in a first processing step. In order to achieve that the oil phases had to be heated to 90° C.

Unfortunately, such a processing is not possible for many types of cosmetic formulations due to limitations in available processing equipment or due to restrictions caused by temperature sensitive ingredients in the formulations. Also for ecological and economical (longer processing, costs for heating) reasons such a way of processing is not optimal.

Moreover, in the Dietz paper it has also been described that all attempts to include Ceramide 3 (Cer NS) in a stable way into water-in-oil emulsions failed. Ceramide 3 (Cer NS) crystals appeared in all formulations after a few days of storage at room temperature even at very low ceramide concentrations of 0.1% by weight in these formulations.

A limitation of stable inclusion of ceramides in formulations just to oil-in-water emulsions is a significant restriction for cosmetic formulators as water-in-oil emulsions are known for their moisturizing benefits especially for dry skin.

Additionally it is even more challenging to include ceramides with N-acyl side chains that are longer than 18 Carbon atoms stable into formulations as these long-chain ceramides tend to crystallize much easier.

However, in order to provide a mixture of ceramides/cholesterol/fatty acids that is able to mimic skin identical lamellar structures, Bouwstra and her group showed that especially such long-chain ceramides are needed (Bouwstra, J. A. et al. J. Lipid Research 1999, 39, 186-196; Bouwstra, J. A. et al. J. Invest. Dermatol. 2002, 118, 606-617; de Jager, M. W. et al. Chem. Phys. Lipids 2003, 124, 123-134)

For all these reasons there is a need to offer the cosmetic and pharmaceutical formulator a composition that is able
1) to mimic skin identical lamellar structures, containing long-chain ceramides (N-acyl side chain longer than 18 carbon atoms) without crystallization of ceramides in the composition upon storage,
2) to be incorporated easily into formulations without the necessity of heating the oil phase or water phase to temperatures above 40° C. Therefore the composition needs to be liquid at room temperature and should have no compatibility problems with anionic, amphoteric or cationic ingredients,
3) to allow the stable inclusion of long-chain ceramides into oil-in-water emulsions as well as into water-in-oil emulsions. In both types of emulsion systems the composition should be able to prevent recrystallization of ceramides upon storage.

Surprisingly, it was found that all three targets can be fulfilled by a suitable design of a skin treatment composition based on nonionic hydrophilic emulsifiers, consistency enhancers, specific ceramide mixtures containing medium and long-chain ceramides, fatty acids and cholesterol. This cholesterol can either be animal derived, be produced by a micro-organism, have a plant origin or be synthetized starting from plant-derived material.

Additionally, it was surprisingly found that inclusion of short chain ceramides (C4 to C8 alkyl chains) did not change the lamellar lipid organization. This inclusion, however, resulted into an increased production of ceramides in the skin.

Therefore the short-chain ceramides surprisingly acted as regulators when the cosmetic composition was applied onto the skin.

The present invention therefore corresponds to liquid and pumpable skin treatment compositions containing (all percentages are given as weight-%):
A) 0.1 to 10% by weight preferred 0.2 to 8% by weight, specially preferred 0.5 to 5% by weight of a mixture consisting of:
   A1) 10 to 80% by weight of a ceramide or of a mixture of at least two ceramides with the proviso that at least one of these ceramides contains a long alkyl, alkenyl or acyl side chains with >18 carbon atoms and that less than 40% by weight has a side chain larger than 26 carbon atoms,
   A2) 10 to 45% by weight of cholesterol,
   A3) 10 to 45% by weight of free fatty acids (C12 to C30 alkyl-, alkenyl-, alkadienyl-, alkatrienyl-, alkapolyenyl chains or combinations thereof;
B) 0.5 to 10% by weight, preferred 1 to 8% by weight, specially preferred 4 to 6% by weight, of a nonionic emulsifier or a nonionic emulsifier mixture with a combined HLB value of 12 to 19;
C) ≧40% by weight of water and optionally;
D) 0.1 to 10% by weight, preferred 0.1 to 6% by weight, specially preferred 1 to 5% by weight, consistency enhancers like glyceryl stearate or C16-C22 alkanols and optionally;
E) auxiliaries and additives
   with the proviso that A) to E) must add up to 100% by weight by weight.

The main advantages of the inventive mixture are:
1) has liquid and pumpable/pourable condition at room temperature i.e. about 20° C. to 30° C. and
2) shows no crystallization of ceramides upon storage for up to 1 year at temperatures between −5° C. and 40° C. and
3) can easily be incorporated into cosmetic formulations without the necessity of heating the oil phase or water phase to temperatures above 40° C., showing no incompatibility effects with anionic, amphoteric or cationic ingredients and
4) makes the stable inclusion of the long-chain ceramides into oil-in-water as well as into water-in-oil emulsions possible by preventing recrystallization of ceramides in these formulations upon storage for up to 1 year at temperatures between 5° C. and 40° C.
5) enables the stable inclusion of specific Ceramides mixtures that are able to mimic skin identical lamellar structures, into oil-in-water as well as into water-in-oil emulsions by preventing recrystallization of ceramides in these formulations upon storage for up to 1 year at temperatures between 5° C. and 40° C.
6) allows for the addition of short chain Ceramides to these specific Ceramides mixtures without losing any of the advantages mentioned above.

In a preferred embodiment of the invention the skin treatment composition contains 0.5 to 5% by weight of the ceramide/cholesterol/free fatty acid mixture A) and 4 to 6% by weight of a nonionic emulsifier or a nonionic emulsifier mixture with a combined HLB value of 12 to 19 and 1 to 5% by weight consistency enhancers like glyceryl stearate or C16 to C22 alkanols and optionally auxiliaries and additives added up to 100% by weight by weight with water.

The Ceramide(s) present in mixture A1) consist of:
1) a sphingoid base with a general structure according to formula 1

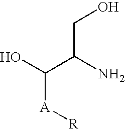

wherein
A is —CH₂—CH₂—, —CH═CH—, or —C(H)OH—CH₂— and
R is a straight chain or branched alkyl group having 10 to 22 carbon atoms which may optionally contain one or more double bonds and/or may optionally be substituted with one or more hydroxyl groups.

Preferably R is either a straight chain alkyl group having 12 to 18 carbon atoms or C(H)OH coupled to a straight chain alkyl group having 11 to 17 carbon atoms.

Most preferred A and R combinations are Phytosphingosine (P), Sphingosine (S), 6-hydroxysphingosine (H) and Sphinganine (Sa).

The above Sphingoid base is coupled via an amide linkage to a fatty acid (both hydroxy and non hydroxy fatty acids) according to formula 2

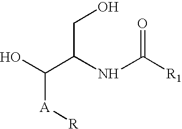

wherein
R₁ is a linear or branched alkyl chain containing 1 to 55 carbon atoms, preferably 3 to 50 carbon atoms and more preferably 5 to 44 carbon atoms.

The alkyl chain may be interrupted by an Oxygen atom or by an internal ester group; may eventually contain one or more double bonds and may eventually be substituted by one or more hydroxyl groups,

or
skinidentical Ceramide(s). These are Ceramides with a composition as above but with a stereochemical configuration, that is identical to the natural ceramides present in the mammalian skin,
or
natural Ceramide(s). These are Ceramides with a composition as above, which are extracted from the mammalian skin and have maintained their original steriochemical configuration during the extraction process,
or
combinations thereof.

In a preferred embodiment of the invention the ceramide/cholesterol/free fatty acid mixture A contains a combination of natural or skin identical ceramides A1) consisting of:
A1a) a content >30% by weight of ceramides with a long alkyl, alkenyl or acyl chain (>C18), while the contents of very long chain (≧C27) acylceramides should be not more than 40% by weight;
A1b) a content of 1 to 30% by weight by weight of ceramides with a short alkyl chain (C4 to C8).

Mixture A1) present in the composition according to the invention consists preferably of:
a combination of at least 2 Ceramides with a long alkyl, alkenyl or acyl sidechain, long meaning more than 18 Carbon atoms;
at least one of these Ceramides should be Ceramide NS, NP, NH with a chain length of 20 to 26 Carbon atoms, and should form at least 40% by weight of Mixture A1);
acylceramides like Ceramide EOS, EOP, EOH should not be present in more than 40% by weight of mixture A1);
part of the Sphingosine, 6-hydroxysphingosine and Phytospingosine derived Ceramides may be exchanged by their Sphinganine derived equivalents;
optionally Ceramides of medium side chain length (16 to 18 Carbon atoms) can be added to mixture A1) to a maximum of 30% by weight;
optionally 1) Ceramides of short side chain length (4 to 8 C atoms) or 2) Sphingoid bases and/or Ceramides with a side chain of 2 Carbon atoms can be added to mixture A1). This to a maximum of 30% by weight (1) and 15% by weight (2) respectively.

In a more preferred embodiment of the invention the ceramides/cholesterol/free fatty acid mixture A contains specific combinations of natural or skin identical ceramides A1, that are able to mimic skin identical lamellar structures (in the following referred to as A1 lam). For this aim Ceramides mixture A1ₗₐₘ, should consist of at least 3, but preferably 4 natural or skin-identical ceramides:
8 to 35% by weight preferably 9 to 15% by weight of one or two Acyl Ceramides with a side chain of ≧27 carbon atoms, wherein up to 45% by weight of the total acyl ceramides can be ceramide 9
≧10% by weight, preferably 25 to 40% by weight of at least one sphingosine based Ceramide NS or AS,
≧10% by weight, preferably 25 to 40% by weight of at least one phytosphingosine based Ceramide NP or AP, preferably Ceramide 3 (Cer NS) optionally up to 30% by weight of natural or skin-identical ceramides with a short alkyl side chain (C4 to C8) can be added.

Most preferred compositions of Mixture A1_{lam.} being able to mimic skin identical lamellar structures consist of:
50 to 70% by weight of Ceramide NS+Ceramide NP in a 45/55 ratio. Both with a side chain length of 20 to 26 C atoms. Upto 20% by weight of the NS+NP may be replaced by Ceramide NH with a similar sidechain length;
14 to 20% by weight of Ceramide EOS+Ceramide EOP in a 6 to 4 ratio. Both with side chain lengths between 27 and 32 C atoms. Upto 20% by weight of the EOS+EOP may be replaced by Ceramide EOH with a similar side chain length;
≧10% by weight, preferably 15% by weight, of Ceramide NP with a medium side chain length of 16 to 18 C atoms. About 30% by weight of the Ceramide NP may be replaced by its Sphingosine [NS], 6-OH Sphingosine [NH] or Sphinganine [NSa] based equivalent;
≧0% by weight, preferably 10% by weight, of Ceramide AP with a side chain length of 16 to 24 C atoms. About 30% by weight of the Ceramide NP may be replaced by its Sphingosine [NS], 6-OH Sphingosine [NH] or Sphinganine [NSa] based equivalent;
optionally about 10% by weight of short chain (Side chain of 4 to 8 C atoms) Ceramide NS+NP in a 1/1 ratio may be added to the A1 mixture. Upto 20% by weight of the NS+NP may be replaced by Ceramide NH or NSa with a similar sidechain length. When using this option the dosages of the medium and long chain Ceramides in mixture A1_{lam.} are reduced proportionally.

The free fatty acids A3) present in the composition according to the invention consists preferably of fatty acids with an alkyl, alkenyl-, alkadienyl-, alkatrienyl-, alkapolyenyl-chain of C12 to C30, most preferably of C18 to C26, or combinations thereof. In a preferred embodiment of the invention behenic acid (C22) is used as free fatty acid component A3).

The nonionic emulsifier or emulsifier mixture B) present in the composition according to the invention include compounds from at least one of the following groups:
addition products of from 2 to 30 mol of ethylene oxide and/or 0 to 5 mol of propylene oxide onto linear fatty alcohols having 8 to 22 carbon atoms, onto fatty acids having 12 to 22 carbon atoms and onto alkylphenols having 8 to 15 carbon atoms in the alkyl group;
C_{12/18}-fatty acid mono- and diesters of addition products of from 1 to 30 mol of ethylene oxide onto glycerol;
glycerol mono- and diesters and sorbitan mono- and diesters of saturated and unsaturated fatty acids having 6 to 22 carbon atoms and ethylene oxide addition products thereof;
alkyl mono- and oligoglycosides having 8 to 22 carbon atoms in the alkyl radical and ethylene oxide addition products thereof;
addition products of from 2 to 200 mol of ethylene oxide onto castor oil and/or hydrogenated castor oil;
partial esters based on linear, branched, unsaturated or saturated C₆₋₂₂-fatty acids, ricinoleic acid, and 12-hydroxystearic acid and glycerol, polyglycerol, pentaerythritol, dipentaerythritol, sugar alcohols (e.g. sorbitol), alkyl glucosides (e.g. methyl glucoside, butyl glucoside, lauryl glucoside), and polyglucosides (e.g. cellulose). Here, the use of partial esters of glycerol, of polyglycerol and of sorbitol is preferred. These are, for example, glyceryl laurate, polyglyceryl-4 laurate and sorbitan laurate;
mono-, di- and trialkyl phosphates;
mixed esters of pentaerythritol, fatty acids, citric acid and fatty alcohol according to DE-11 65 574 and/or mixed esters of fatty acids having 6 to 22 carbon atoms, methylglucose and polyols, preferably glycerol or polyglycerol.

The nonionic emulsifier or emulsifier mixture B) present in the composition according to the invention consists preferably of a nonionic ethoxylated emulsifier or a mixture of nonionic ethoxylated emulsifiers with a total HLB value of 12 to 19, most preferably with a HLB value of 14 to 18.

The HLB value for an ethoxylated emulsifier Eᵢ is typically calculated by the formula (1)

$${HLB}_{i} = \frac{{E}_{i}}{5} \qquad ( 1 )$$

with Eᵢ being the weight percentage of ethylene oxide groups in the ethoxylated emulsifier i. When an emulsifier mixture is used, the total HLB value (HLBₘᵢₓₜᵤᵣₑ) is calculated as the simple linear mass weighted average as given by formula (2)

$${HLB}_{mixture} = {\sum }_{i} [ ( \frac{{m}_{i}}{{m}_{total}} ) \cdot {HLB}_{i} ] \qquad ( 2 )$$

with (mᵢ/mₜₒₜₐₗ) being the mass fraction of the emulsifier i.

This type of calculation of the HLB value of nonionic emulsifiers and emulsifier mixtures is based on empirical equations derived by Griffins (W. C. Griffin, J. Soc. Cosmet. Chem., 1949, 1, 311 and W. C. Griffin, J. Soc. Cosmet. Chem., 1954, 5, 249). Alternative calculations of the HLB value are summarized in several text books on emulsion science (e.g. B. P. Binks (edt.), Modern Aspects of Emulsion Science, The Royal Society of Chemistry, Cambridge, 1998).

In a preferred embodiment of the invention Ceteareth-25 (HLB=16.2) is used as hydrophilic nonionic emulsifier component B).

As auxiliaries and additives G according to the invention all auxiliaries and additives customary in cosmetic and pharmaceutical applications and known to the person skilled in the art can be used. These include, for example, additional consistency regulators, thickeners, waxes, UV photoprotective filters, antioxidants, hydrotropes, preservatives, perfume oils, dyes and additional biogenic active ingredients as described for example in DE 10 2005 011 785.6.

As explained above, the liquid and pumpable skin treatment composition can easily be incorporated in cosmetic, dermatological or pharmaceutical formulations in a cold process.

The cosmetic or dermatological formulation can be an aqueous solution, a water-in-oil (W/O) emulsion, an oil-in-water (O/W) emulsion, an aqueous or a water-alcohol gel, a wet-wipe or an aerosol. In case a hot process is needed to prepare the desired formulation type, e.g. solid sticks or wax containing emulsions, the liquid and pumpable skin treatment composition can also be used for such type of hot processing.

The cosmetic or dermatogical formulation is preferably a W/O or an O/W emulsion that contains 1 to 50% by weight of an oil phase and 47 to <99% by weight water, with respect to the weight of the whole formulation. The oil phase can contain all types of cosmetic emollients known to the person skilled in the art. The emulsions are stabilized by all types of emulsifiers, stabilizing polymers and thickeners known to the person skilled in the art. Examples for such emollients, emulsifiers, stabilizing polymers and thickeners are described in DE 10 2005 011 785.6.

Therefore, the invention further provides cosmetic, dermatological or pharmaceutical preparations which comprise a ceramide containing skin treatment composition according to the invention.

In an embodiment of the invention the skin treatment composition is included in a topical skin treatment formulation with an effective amount between 0.001 and 20% by weight.

In a preferred embodiment of the invention the skin treatment composition is included in a topical skin treatment formulation with an effective amount between 0.05 and 10% by weight in order to maximize benefits at minimum costs.

Besides the skin treatment composition other specific skin-benefit actives such as anti-ageing actives, moisturizers, sunscreens, skin lightening agents, skin tanning agents may also be included.

Typical additional bioactive compounds are:
vitamins and derivatives thereof like tocopherol, ascorbic acid, niacinamide, retinol, panthenol;
antioxidants like alpha-lipoic acid, Coenzyme Q10, idebenone, polyphenolics, flavonoids, stilbens, hydroxystilbens, xanthones or isoflavones;
anti-inflammatories like bisabolol, allantoin, phytantriol, phytosphingosine, sphingosine;
moisturising agents, amino acids, hyaluronic acid, polyglutamic acid, trimethylglycine, myoinositol, pyroglutamatic acid, taurine, guanidine and hydroxy acids;
anti-ageing actives like peptides, modified peptides, proteinhydrolysates, lysophospholipids, beta-glucans, creatine, alpha hydroxy acids, beta hydroxy acids, plant extracts or microbial extracts;
anti-cellulite agents like caffeine or carnitine;
skin whitening agents like kojic acid, arbutin, vitamin C and derivatives, hydroquinone, creatinine;
skin tanning agents like dihydroxyacetone, erythrulose or N-acetyl tyrosin.

In addition, auxiliaries and additives customary in cosmetic and pharmaceutical applications and known to the person skilled in the art can be used. These include, for example, co-emulsifiers, consistency regulators, thickeners, waxes, organic and inorganic UV filters, pigments, buffers, hydrotropes, deodorant and antiperspirant active ingredients, insect repellents, antioxidants, self-tanning agents, preservatives, perfume oils and dyes (as described for example in DE 10 2005 011 785.6).

In addition to topical skin treatment formulations the skin treatment composition can also be incorporated into hair care formulations such as, for example, shampoos and conditioners, where it can show a stimulating effect on the scalp performance.

Another embodiment of the invention is therefore the use of the liquid and pumpaple skin treatment composition for hair care applications.

The liquid and pumpable skin treatment compositions according to the invention can also be used in so-called “wash-off” products, e.g., body wash formulations or bath or shower gels.

Therefore, another embodiment of the invention is the use of the liquid an pumpable skin care treatment in “wash-off” applications.

To prepare formulations containing the liquid and pumpable skin treatment composition according to the present invention, the usual manner for preparing such formulations may be employed. The formulations containing the inventive skin treatment composition may be packaged in any suitable manner such as in a jar, a bottle, tube, roll-ball, or the like, in the conventional manner.

Skin is subject to deterioration through environmental abuse (wind, air conditioning, central heating, pollution, sun exposure etc.) accompanied by the passage of time (chronological ageing, dermatological disorders, hormonal changes). These factors lead to decreased skin performance that manifest themselves among others by dry, rough and rigid skin. These effects are observable in vivo by parameters like skin moisture (corneometer measurement), barrier function (transepidermal waterloss measurement) or elasticity (cutometer measurement). Concomitantly to the physiologically observable parameters, there are also processes involved on a molecular level:
expression of genes involved in differentiation and built up of structural and enzymatic components of the skin barrier (e.g. Fillagrin, Loricrin, Involucrin, Transglutaminase);
expression of genes involved in the lipid components of the barrier, especially sphingolipid biosynthesis (e.g. Serine-Palmitoyl Transferase and Glucosyl-Ceramide Transferase);
expression of genes responsible for the formation of tight junctions as a separation layer between the viable epidermis and the cornified Stratum Corneum (e.g. claudin-1) and the water transport in the viable layers of the epidermis (e.g. aquaporin-3).

Cosmetic products which treat or delay the visible signs of ageing and improve the environmental protection should increase the expression marker of genes mentioned above leading to a measurable improvement of classical skin performance parameters.

Dermatological studies revealed that the application of cosmetic formulations containing the skin treatment compositions showed such significant increase of these expression markers.

### EXAMPLES

The following example emulsions serve to illustrate the subject-matter of the invention in more detail without limiting it to these examples. The concentration data in all examples are given as % by weight.

### Examples for Liquid and Pumpaple Skin Treatment Compositions

The examples STC 1 to 10 illustrate liquid and pumpable skin treatment compositions according to the invention. The skin treatment compositions are prepared by heating the oil phase to 90 to 120° C. depending on the Ceramides present (in order to be above the melting point of the dosed ceramides) and heating the water phase to 90° C. Then both phases are combined and homogenized for a short time. Liquid and pumpable skin treatment compositions are obtained that can easily be incorporated into e.g. cosmetic formulations.

| | STC 1 | STC 2 | STC 3 | STC 4 | STC 5 |
|---|---|---|---|---|---|
| Oil Phase | | | | | |
| Ceteareth-25 | 6.00% | 5.00% | 4.00% | 6.00% | 4.00% |
| Glyceryl Stearate | | | 2.00% | | 2.00% |
| Stearyl Alcohol | 2.00% | 3.00% | 2.00% | 2.00% | 2.00% |
| Behenic Acid | 0.51% | 0.51% | 0.51% | 0.77% | 0.77% |
| Cholesterol³⁾ | 0.50% | 0.50% | 0.50% | 0.75% | 0.75% |
| Ceramide 1 (EOS) C₂₇₋₃₂⁴⁾ | 0.094% | 0.094% | 0.094% | 0.142% | 0.142% |
| Ceramide 9 (EOP) C₂₇₋₃₂⁴⁾ | 0.058% | 0.058% | 0.058% | 0.088% | 0.088% |
| Ceramide 2 (NS) C₂₂⁵⁾ | 0.276% | 0.276% | 0.276% | 0.414% | 0.414% |
| Ceramide 3 (NP) C₂₂⁵⁾ | 0.329% | 0.329% | 0.329% | 0.494% | 0.494% |
| Ceramide 3 (NP) C₁₆⁵⁾ | 0.154% | 0.154% | 0.154% | 0.231% | 0.231% |
| Ceramide 6 (AP) C₁₈⁵⁾ | 0.089% | 0.089% | 0.089% | 0.134% | 0.134% |
| Ceramide 3 (NP) C₆⁵⁾ | 0.05% | 0.05% | 0.05% | 0.05% | 0.05% |
| [= n-Hexanoyl | | | | | |
| Phytosphingosine] | | | | | |
| Ceramide 2 (NS) C₆⁵⁾ | 0.05% | 0.05% | 0.05% | 0.05% | 0.05% |
| [= n⁻Hexanoyl | | | | | |
| Sphingosine] | | | | | |
| Oxynex ® LM¹⁾ | 0.10% | 0.10% | 0.10% | 0.10% | 0.10% |
| Water Phase | | | | | |
| Glycerin | 3.00% | 3.00% | 3.00% | 3.00% | 3.00% |
| Euxyl ® K 300²⁾ | 1.00% | 1.00% | 1.00% | 1.00% | 1.00% |
| Water | ad | ad | ad | ad | ad |
| | 100% | 100% | 100% | 100% | 100% |
| Consistency | thin | thin | liquid | thin | liquid |
| | liquid | liquid | | liquid | |
| Crystals | No | no | no | no | no |
| Stability | Stable | stable | stable | stable | stable |
| | STC 6 | STC 7 | STC 8 | STC 9 | STC 10 |
| Oil Phase | | | | | |
| Ceteareth-25 | 6.00% | 6.00% | 5.00% | 4.00% | 6.00% |
| Glyceryl Stearate | | | | 2.00% | |
| Stearyl Alcohol | 2.00% | 2.00% | 3.00% | 2.00% | 2.00% |
| Behenic Acid | 0.51% | 0.51% | 0.51% | 0.51% | 0.77% |
| Cholesterol | 0.15% | 0.30% | 0.30% | 0.15% | 0.23% |
| Ceramide 1 (EOS) C₂₇₋₃₂⁴⁾ | 0.094% | 0.094% | 0.094% | 0.094% | 0.142% |
| Ceramide 9 (EOP) C₂₇₋₃₂⁴⁾ | 0.058% | 0.058% | 0.058% | 0.058% | 0.088% |
| Ceramide 2 (NS) C₂₂⁵⁾ | 0.276% | 0.276% | 0.276% | 0.276% | 0.414% |
| Ceramide 3 (NP) C₂₂⁵⁾ | 0.329% | 0.329% | 0.329% | 0.329% | 0.494% |
| Ceramide 3 (NP) C₁₆⁵⁾ | 0.154% | 0.154% | 0.154% | 0.154% | 0.231% |
| Ceramide 6 (AP) C₁₈⁵⁾ | 0.089% | 0.089% | 0.089% | 0.089% | 0.134% |
| Ceramide 3 (NP) C₆⁵⁾ | 0.05% | 0.05% | 0.05% | 0.05% | 0.05% |
| [= n-Hexanoyl | | | | | |
| phytosphingosine] | | | | | |
| Ceramide 2 (NS) C₆⁵⁾ | 0.05% | 0.05% | 0.05% | 0.05% | 0.05% |
| [= n⁻Hexanoyl Sphingosine] | | | | | |
| Oxynex ® LM¹⁾ | 0.10% | 0.10% | 0.10% | 0.10% | 0.10% |
| Water Phase | | | | | |
| Glycerin | 3.00% | 3.00% | 3.00% | 3.00% | 3.00% |
| Euxyl ® K 300²⁾ | 1.00% | 1.00% | 1.00% | 1.00% | 1.00% |
| Water | ad | ad | ad | ad | ad |
| | 100% | 100% | 100% | 100% | 100% |
| Consistency | thin | thin | thin | thin | liquid |
| | liquid | liquid | liquid | liquid | |
| Crystals | no | no | no | no | no |
| Stability | stable | stable | stable | stable | stable |

¹⁾Oxynex ® LM (Merck): or alternative antioxidant mixture
²⁾Euxyl ® K 300 (Schulke & Mayr): preservative mixture
³⁾ Cholesterol either animal or plant based
⁴⁾C₂₇₋₃₂ means with a fatty acid side chain length of 27 to 32 Carbon atoms
⁵⁾Cₙ means with a fatty acid chain length of about n Carbon atoms

All compositions are stable for at least one year at temperatures from −5 to 40° C. No ceramide crystals can be found in this period for all tested temperatures. This illustrates the excellent storage stability of the skin treatment compositions.

### Examples for Ceramide/Cholesterol/Free Fatty Acid Mixtures Able to Mimic Skin Identical Lamellar Structures

### Summary Materials and Methods SAXD Experiments

### Methods

All methods used in the SAXD diffraction studies were derived from the work of Prof. Bouwstra and her group. (de Jager et al., J. Lipid Res. 2004, 45, 923-932; de Jager et al., J. Lipid Res. 2005, 46, 2649-2656).

### A1ₗₐₘ Mixture Compositions [Sum Long+Mid Chain Ceramides is 100%]

### Natural Ceramides

Pig CER (1-6) mixture, composition conform Bouwstra et al., J. Lipid Res., 1998, 39, 186 to 196. (Please Note: Composition in % mol instead of % by weight)
Human Cer mixture, conposition conform: Bouwstra et al., J. Invest. Dermatol., 2002, 118, 606-617. (Please Note: Composition in % mol instead of % by weight)

### Skin Identical Ceramides

### SynthCER II₁, Composition in mol % (De Jager et al., J. Lipid Res. 2005, 46, 2649-2656)

Ceramide 1 (C₃₀) 15; Ceramide 2 (C₂₄) 51; Ceramide 3 (C₂₄) 16; Ceramide 3 (C₁₆) 9; Ceramide 4 (C₂₄) 4; Ceramide 6 (C₂₄) 5

### SynthCER II₂, Composition in mol % (De Jager et al., J. Lipid Res. 2005, 46, 2649-2656)

Ceramide 1 (C₃₀) 10; Ceramide 9 (C₃₀) 5; Ceramide 2 (C₂₄) 51; Ceramide 3 (C₂₄) 16; Ceramide 3 (C₁₆) 9; Ceramide 4 (C₂₄) 4; Ceramide 6 (C₂₄) 5
Scer6A=STC, This patent

| | Scer6A | Scer6A | Scer6A + |
|---|---|---|---|
| | % mol | % w | C2c6C3c6 |
| Ceramide 1 (EOS) C₂₇₋₃₂⁴⁾ | 6.25 | 9.4% | 9.4% |
| Ceramide 9 (EOP) C₂₇₋₃₂⁴⁾ | 3.75 | 5.8% | 5.8% |
| Ceramide 2 (NS) C₂₂⁵⁾ | 28.5 | 27.6% | 27.6% |
| Ceramide 3 (NP) C₂₂⁵⁾ | 33.0 | 32.9% | 32.9% |
| Ceramide 3 (NP) C₁₆⁵⁾ | 18.6 | 15.4% | 15.4% |
| Ceramide 6 (AP) C₁₈⁵⁾ | 9.9 | 8.9% | 8.9% |
| Ceramide 3 (NS) C₆⁵⁾ | 0.0 | 0.0% | 5.0% |
| [=n-Hexanoyl Phytosphingosine] | | | |
| Ceramide 2 (NS) C₆⁵⁾ | 0.0 | 0.0% | 5.0% |
| [=n⁻Hexanoyl Sphingosine] | | | |

⁴⁾C₂₇₋₃₂ means with a fatty acid side chain length of 27 to 32 Carbon atoms
⁵⁾Cₙ means with a fatty acid chain length of about n Carbon atoms

### A2 Cholesterol Compositions

### Cholesterol

If not mentioned specifically otherwise, animal based cholesterol HP was used, which was supplied by Solvay. Pharmaceuticals, Veenendaal.

### Plant Derived, Semisynthetic Cholesterol

Plant cholesterol provided by Degussa Care Specialties or Syntechol supplied by Sigma.

### A3 Free Fatty Acids

FFA mix, composition conform (de Jager et al., J. Lipid Res. 2004, 45, 923-932;
Fatty acids C16:0, C18:0, C20:0, C22:0, C23:0, C24:0 and C26:0 were mixed at molar ratios of respectively 1.3, 3.3, 6.7, 41.7, 5.4, 36.8 and 4.7% by weight respectively;
FFA, linoleic acid As above but C23:0 replaced by linoleic acid;
FFA, arachidonic acid As above but C23:0 replaced by Arachidonic acid.

### Behenic Acid [C16 to C24]

Plant derived free fatty acid mixture containing mainly of Behenic acid' (±85% mol). Further C16:0, C18:0, C20:0, C21:0, C23:0 and C24:0 are present.

### Preparation of the Mixtures for SAXD Diffraction

Samples were prepared with the ceramide mixture described above. The CER mixture was mixed with cholesterol and free fatty acids in equimolar ratio, if not specifically mentioned otherwise. Appropriate amounts of individual lipids dissolved in chloroform:methanol (2:1) were combined to yield mixtures of approximately 1.5 mg total weight at the desired composition with a total lipid concentration of 7 mg/ml. A Camag Linomat IV was used to apply the lipid mixtures on mica. This was done at a rate of 4.3 μl/min under a continuous nitrogen stream. The samples were equilibrated for 10 minutes at the appropriate temperature of 65° C. and subsequently hydrated with an acetate buffer of pH 5.0. Finally, the samples were homogenized by 10 successive freeze-thawing cycles between −20° C. and room temperature, during which the samples were stored under gaseous argon.

### Small Angle X-ray Diffraction (SAXD)

All measurements were performed at the European Synchrotron Radiation Facility ESRF, Grenoble) using station BM26B. The X-ray wavelength and the sample-to-detector distance were 1.24 Å and 1.7 m, respectively. Diffraction data were collected with a two-dimensional multiwire gas-filled area detector. The spatial calibration of this detector was performed using silver behenate. The samples were mounted in a temperature-controlled sample holder with mica windows. The diffraction patterns of the lipid mixtures were obtained at room temperature for a period of 10 minutes.

Small angle X-ray diffraction provides information about the larger structural units in the sample, namely the repeat distance of a lamellar phase. The scattering intensity I (in arbitrary units) was measured as a function of the scattering vector q (in reciprocal nm). The latter is defined as q=(4π sin θ)/λ, in which θ is the scattering angle and λ is the wavelength. From the positions of a series of equidistant peaks (qₙ), the periodicity, or d-spacing, of a lamellar phase was calculated using the equation d=2nπ/qₙ, n being the order number of the diffraction peak.

### Example 1

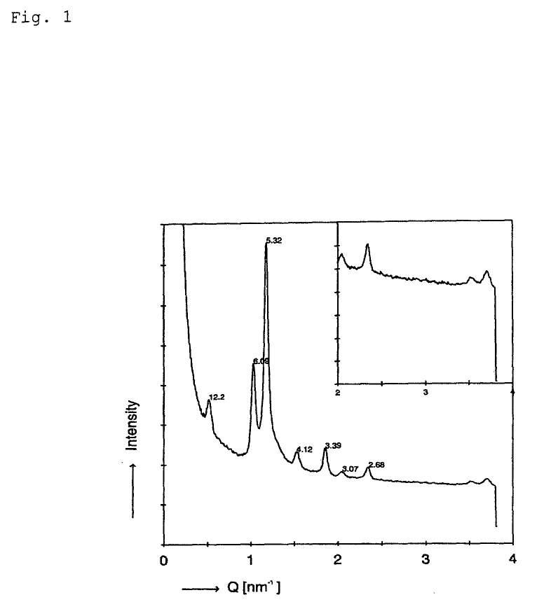

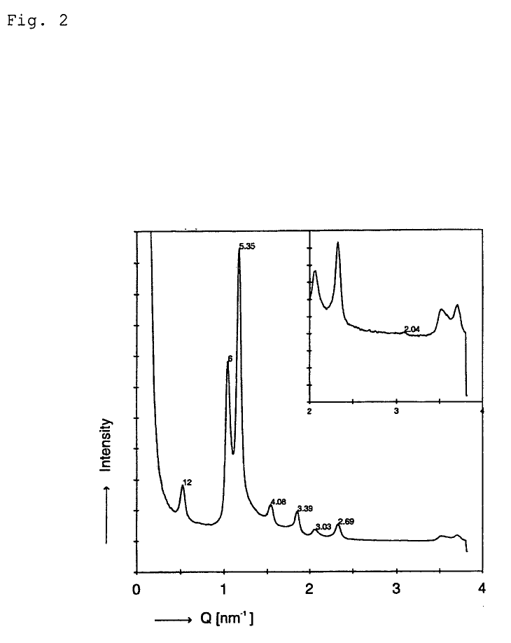

### SAXD Patterns with as A1: SynthCerII_{I} Mixture [FIG. 1] and Scer6 Mixture [FIG. 2]

Just as published by Prof. Bouwstra and her group SynthCerII_{I} and Scer6 show SAXD patterns similar to human Cer mixture and pig Cer [1-6] mixture. Compare FIG. 1 and FIG. 2.

### Example 2

### SAXD Patters with as A1 lₐₘ Mixture

Scer6

| | |
|---|---|
| Scer6 | +10% short chain Ceramides NS + NP |
| Scer6 | FFA_{linoleic acid} |

The SAXD patterns show that the formation of the lamellar LPP and SPP structures are not affected by the addition of short chain Ceramides.

Despite the presence of Linoleic acid in the fatty acid mixture the LPP and SPP structures are still formed. The same is true for Arachidonic acid (not shown).

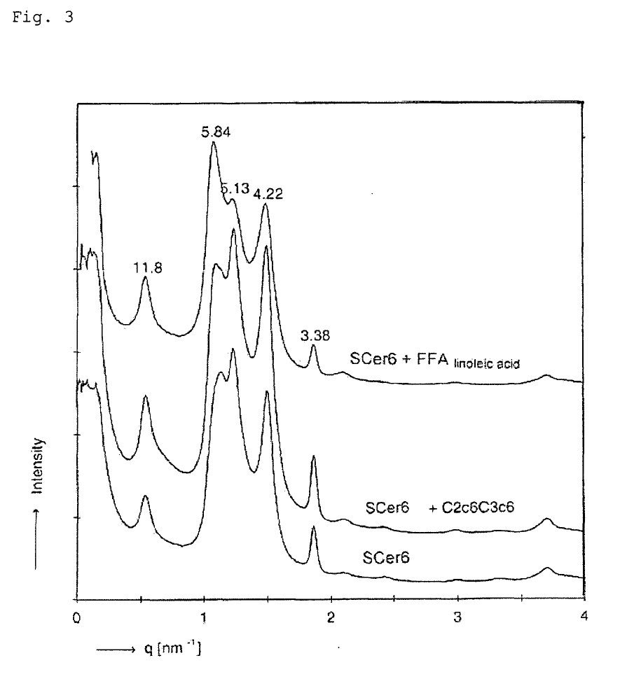

### Compare FIG. 3

### Examples for Cosmetical Skin Care Formulations

These examples illustrate the easy use of the skin treatment compositions (STC 1, STC 2 and STC 4) in the preparation of cosmetic skin treatment formulations. The skin treatment compositions can be added to the water phase at room temperature. Depending on the processing the water phase can be processed at room temperature or it can be heated, e.g. to 80° C. Therefore the skin treatment compositions can be used in cold and hot processing of 0/W emulsions (formulations 1 to 4 and 5 to 8) and for the processing of W/O emulsions (formulations 9 to 12).

All formulations have been successfully tested on stability for at least six months at temperatures from −5° C. to 40° C., additionally for 3 months at 45° C. Freeze stability was successfully tested in 3 freeze-thaw cycles between room temperature and −15° C.

### Formulation Examples 1 to 4

### Cold Processed O/W Emulsions

Processing: Phases A and B are combined at room temperature, the emulsion is homogenized. Additional phases are added afterwards.

| | 1 | 2 | 3 | 4 |
|---|---|---|---|---|
| A (Oil Phase) | | | | |
| Bis-PEG/PPG-16/16 PEG/ | 2.00% | 2.00% | | 2.00% |
| PPG-16/16 Dimethicone, | | | | |
| Caprylic/Capric | | | | |
| Triglyceride³⁾ | | | | |
| Sorbitan Laurate, | | | 1.50% | |
| Polyglyceryl-4 Laurate, | | | | |
| Dilauryl Citrate⁴⁾ | | | | |
| Caprylic/Capric | 5.00% | | | |
| Triglyceride | | | | |
| Ethylhexyl Stearate | 3.30% | 4.00% | | 5.00% |
| Ethylhexyl Palmitate | | 4.00% | 7.00% | 5.00% |
| Diethylhexyl Carbonate | 3.30% | 5.50% | | 7.50% |
| Mineral Oil | 5.00% | | | |
| Octyldodecanol | | 4.00% | | |
| Decyl Cocoate | | | 3.50% | |
| Cetearyl Isononaoate | | | 3.00% | |
| Tocopheryl Acetate | 1.00% | 0.50% | | 0.50% |
| B (Water Phase) | | | | |
| Skin Treatment | 5.00% | | 5.00% | 5.00% |
| Composition | | | | |
| 1 (STC 1) | | | | |
| Skin Treatment | | 5.00% | | |
| Composition | | | | |
| 2 (STC 2) | | | | |
| Water | 60.80% | 68.20% | 75.5% | 70.80% |
| Glycerin | 2.00% | 3.00% | 3.00% | 3.00% |
| Ethanol | 10.0% | | | |
| Allantoin | 0.10% | 0.10% | | |
| Panthenol | 1.00% | 0.50% | | |
| Creatine | | 0.30% | | |
| Carbomer dispersion 1⁵⁾ | | | 1.40% | |
| Carbomer dispersion 2⁶⁾ | | 1.90% | | |
| Carbomer dispersion 3⁷⁾ | | | | 1.00% |
| Xanthan Gum | | 0.10% | 0.10% | |
| Polyacrylamide, C13-14 | 1.50% | | | |
| Isoparaffin, Laureth-7⁸⁾ | | | | |
| Polysorbate 80 | | 0.20% | | 0.20% |
| C | | | | |
| Sodium Hydroxide (10% in | | 0.70% | q.s. | 0 |
| water) | | | | |
| Z | | | | |
| Preservative, Parfum | q.s. | q.s. | q.s. | q.s. |
| Consistency | Lotion | Low | Lotion | Sprayable |
| | | viscous | | Lotion |
| | | lotion | | |

³⁾ABIL ® Care 85 (Degusssa)
⁴⁾TEGO ® Care LTP (Degussa)
⁵⁾Carbomer dispersion 1 consists of 10% TEGO ® Carbomer 140 (Degussa) and 10% TEGO ® Carbomer 141 (Degussa) dispersed in Ethylhexyl Palmitate
⁶⁾Carbomer dispersion 2 consists of 15% TEGO ® Carbomer 141 (Degussa) dispersed in Ethylhexyl Stearate
⁷⁾Carbomer dispersion 3 consists of 10% TEGO ® Carbomer 140 (Degussa) and 10% TEGO ® Carbomer 341 ER (Degussa) dispersed in Ethylhexyl Palmitate
⁸⁾Sepigel ® 305 (Seppic)

### Formulation Examples 5 to 8

### Hot Processed O/W Emulsions

Processing: Phases A) and B) are heated separately to approx. 80° C. Phase A) is added to phase B) with stirring, a homogenization step follows. The emulsions are cooled to 60° C. and phase C) is added, the emulsion is homogenized for a short time. All other phases are added below 40° C.

| | 5 | 6 | 7 | 8 |
|---|---|---|---|---|
| A (Oil Phase) | | | | |
| Bis-PEG/PPG-16/16 | 1.00% | | | |
| PEG/PPG-16/16 | | | | |
| Dimethicone, | | | | |
| Caprylic/Capric | | | | |
| Triglyceride³⁾ | | | | |
| Ceteareth-25 | 1.00% | | | |
| Polyglyceryl-3 | | 3.00% | | |
| Methylglucose | | | | |
| Distearate⁹⁾ | | | | |
| Glyceryl Stearate | | | 1.50% | |
| Citrate¹⁰⁾ | | | | |
| Ceteareth-15, Glyceryl | | | | 2.50% |
| Stearate¹¹⁾ | | | | |
| Glyceryl Stearate | 5.00% | 0.50% | | |
| Stearyl Alcohol | 2.00% | 1.00% | | 1.00% |
| Ethylhexyl Palmitate | 6.00% | 4.00% | 5.00% | |
| Diethylhexyl Carbonate | 6.00% | 12.00% | 6.00% | |
| Caprylic/Capric | 6.00% | 3.00% | | |
| Triglyceride | | | | |
| Isopropyl Palmitate | | | 2.50% | |
| Ethylhexyl Stearate | | | | 5.20% |
| Cetyl Ricinoleate | | | | 3.00% |
| Isocetyl Palmitate | | | | 2.00% |
| Dimethicone | | | | 0.50% |
| Tocopheryl Acetate | | 0.50% | | |
| B (Water Phase) | | | | |
| Skin Treatment | 5.00% | | | 5.00% |
| Composition | | | | |
| 1 (STC 1) | | | | |
| Skin Treatment | | 5.00% | 5.00% | |
| Composition | | | | |
| 4 (STC 4) | | | | |
| Water | 66.30% | 66.60% | 75.10% | 76.40% |
| Glycerin | 3.00% | 3.00% | 3.00% | 3.00% |
| Panthenol | | | 0.50% | |
| C | | | | |
| Carbomer dispersion 4¹²⁾ | 0.50% | 1.00% | | |
| Carbomer dispersion 5¹³⁾ | | | 1.00% | 1.00% |
| D | | | | |
| Sodium Hydroxide | 0.20% | 0.40% | 0.40% | 0.40% |
| (10% in water) | | | | |
| Z | | | | |
| Preservative, Parfum | q.s. | q.s. | q.s. | q.s. |
| Consistency | Cream | Cream | Lotion | Low |
| | | | | viscous |
| | | | | lotion |

⁹⁾TEGO ® Care 450 (Degusssa)
¹⁰⁾AXOL ® C 62 (Degussa)
¹¹⁾TEGO ® Care 215 (Degusssa)
¹²⁾Carbomer dispersion 4 consists of 20% TEGO ® Carbomer 134 (Degussa) dispersed in Isopropyl Palmitate
¹³⁾Carbomer dispersion 5 consists of 20% TEGO ® Carbomer 141 (Degussa) dispersed in Ethylhexyl Palmitate

### Formulation Examples 9 to 12

### W/O Emulsions

Processing: Heat phase A) to approx. 80° C. Phase B) is added while stirring, a homogenization step follows. The emulsions are cooled to 30° C. and phase C) is homogenized again for a short time.

| | 9 | 10 | 11 | 12 |
|---|---|---|---|---|
| A (Oil Phase) | | | | |
| Polyglyceryl-4 | 3.00% | 2.00% | 3.00% | |
| Diisostearate/ | | | | |
| Polyhydroxystearate/ | | | | |
| Sebacate¹⁴⁾ | | | | |
| Diisostearoyl | | 1.00% | | |
| Polyglyceryl-3 Dimer | | | | |
| Dilinoleate¹⁵⁾ | | | | |
| Cetyl PEG/PPG-10/1 | | | | 2.00% |
| Dimethicone¹⁶⁾ | | | | |
| Microcrystalline Wax | 0.10% | 0.10% | 0.10% | 0.50% |
| Hydrogenated Castor Oil | 0.10% | 0.10% | 0.10% | 0.50% |
| Diethylhexyl Carbonate | 7.00% | 7.00% | | |
| Triisostearin | 3.00% | | | |
| Cylcoemethicone | 7.00% | | | |
| C12-15 Alkyl Benzoate | | 7.00% | | |
| Caprylic/Capric | | 6.80% | | |
| Triglyceride | | | | |
| Paraffinum Perliquidum | | | 8.90% | 10.50% |
| (30 mPas) | | | | |
| Ethylhexyl Palmitate | | | 8.90% | 10.50% |
| B (Water Phase) | | | | |
| Skin Treatment | 5.00% | | | 5.00% |
| Composition | | | | |
| 1 (STC 1) | | | | |
| Skin Treatment | | 2.00% | 2.00% | |
| Composition | | | | |
| 2 (STC 4) | | | | |
| Water | 69.80% | 69.50% | 72.50% | 67.50% |
| Glycerin | 3.00% | 3.00% | 3.00% | 3.00% |
| Creatine | 1.00% | | | |
| Magnesium Sulfate | 1.00% | 1.50% | 1.50% | |
| Heptahydrate | | | | |
| Sodium Chloride | | | | 0.50% |
| Z | | | | |
| Preservative, Parfum | q.s. | q.s. | q.s. | q.s. |
| Consistency | Lotion | Lotion | Lotion | Lotion |

¹³⁾ISOLAN ® GPS (Degusssa)
¹⁴⁾ISOLAN ® PDI (Degusssa)
¹⁵⁾ABIL ® EM 90 (Degusssa)

1. A liquid and pumpable skin treatment composition comprising:
A) 0.1 to 10% by weight of a mixture consisting of
A1) 10 to 80% by weight of a mixture of at least two ceramides with the proviso that at least one of these ceramides contains a long alkyl, alkenyl or acyl side chain with 20 to 26 carbon atoms and that less than 40% by weight has a side chain larger than 26 carbon atoms;
A2) 10 to 45% by weight of cholesterol,
said cholesterol can be animal derived, be produced by a micro-organism, have a plant origin or be synthesized starting from a starting material that is derived from plants or produced by a micro-organism; and
A3) 10 to 45% by weight of free fatty acids including C12 to C30 alkyl-, alkenyl, alkadienyl, alkatrienyl or alkapolyenyl chains or combinations thereof;
B) 0.5 to 10% by weight of a nonionic emulsifier or a nonionic emulsifier mixture with a combined HLB value of 12 to 19; and
C) ≧40% by weight of water
with the proviso that at least A) to C) adds up to 100% by weight.

2. A liquid and pumpable skin treatment composition according to claim 1 containing:
A) 0.1 to 10% by weight of a mixture that mimics skin identical lamellar structures consisting of:
A1) 10 to 80% by weight of a mixture of skin identical ceramides consisting of
A1a) a content >30% by weight of ceramides with a long alkyl, alkenyl or acyl chain including greater than 18 carbon atoms, while the content of very long chain acylceramides having greater than 26 carbon atoms is not more than 40% by weight;
A1b) a content of 1 to 30% by weight of ceramides with a short alkyl chain having from 4 to 8 carbon atoms.

3. A liquid and pumpable skin treatment composition according to claim 1 containing:
A) 0.1 to 10% by weight of a mixture that mimics skin identical lamellar structures consisting of specific combinations of natural or skin identical ceramides
A1) that consists of at least 3 natural or skin-identical ceramides:
8 to 35% by weight of one or two Acyl Ceramides with a side chain ≧27 carbon atoms, wherein up to 45% by weight of the total acyl ceramides are ceramide 9
≧10% by weight of at least one phytosphingosine based Ceramide NP or AP, and optionally
up to 30% by weight of natural or skin-identical ceramides with a short alkyl side chain having from 4 to 8 carbon atoms.

4. The liquid and pumpable skin treatment composition according to claim 1 containing 0.2 to 8% by weight of the ceramide/cholesterol/free fatty acid mixture A.

5. The liquid and pumpable skin treatment composition according to claim 1 containing 1 to 8% by weight of said nonionic emulsifier or said nonionic emulsifier mixture with the combined HLB value of 12 to 19.

6. The liquid and pumpable skin treatment composition according to claim 20 containing 0.1 to 6% by weight of said consistency enhancers.

7. The liquid and pumpable skin treatment composition according to claim 1 containing 0.5 to 5% by weight of the ceramide/cholesterol/free fatty acid mixture A), 4 to 6% by weight of said nonionic emulsifier or said nonionic emulsifier mixture with the combined HLB value of 12 to 19, 1 to 5% by weight of consistency enhancers, and optionally auxiliaries and additives added up to 100% by weight with water.

8. The liquid and pumpable skin treatment composition according to claim 1 in which the ceramide mixture
A1) consists of a combination of ceramides that mimics skin identical lamellar structures:
50 to 70% by weight of Ceramide NS+Ceramide NP in a 45/55 ratio; wherein up to 20% by weight of the NS+NP may be replaced by Ceramide NH with a similar sidechain length;
14 to 20% by weight of Ceramide EOS+Ceramide EOP in a 6 to 4 ratio; wherein 20% by weight of the EOS+EOP may be replaced by Ceramide EOH with a similar side chain length;
≧10% by weight of Ceramide NP with a medium side chain length of 16 to 18 C atoms; wherein up to 30% by weight of the Ceramide NP may be replaced by its Sphingosine [NS], 6-OH Sphingosine [NH] or Sphinganine [NSa] based equivalent;
≧0% by weight of Ceramide AP with a side chain length of 16 to 24 C atoms, wherein up to 30% by weight of the Ceramide NP may be replaced by its Sphingosine [NS], 6-OH Sphingosine [NH] or Sphinganine [NSa] based equivalent;
≧0% by weight of Ceramide NS+NP in a 1/1 ratio.

9. The liquid and pumpable skin treatment composition according to claim 1 in which the free fatty acid component A3 is selected from a group of fatty acids with an alkyl or alkenyl chain of C14 to C28.

10. The liquid and pumpable skin treatment composition according to claim 1 wherein the free fatty acid component A3) is behenic acid.

11. The liquid and pumpable skin treatment composition according to claim 1 wherein the nonionic emulsifier or emulsifier mixture B) consists of a nonionic emulsifier or nonionic emulsifier mixture with a total HLB value of 14 to 18.

12. The liquid and pumpable skin treatment composition according to claim 1 wherein the nonionic emulsifier component B) is Ceteareth-25.

13. A cosmetic, dermatological or pharmaceutical skin treatment formulation containing 0.001 to 20 by weight of a skin treatment composition according to claim 1.

14. The cosmetic, dermatological or pharmaceutical skin treatment formulation according to claim 13 further containing at least one component with additional skin benefit actives selected from the group consisting of vitamins, anti-oxidants, anti-inflammatories, anti-aging actives, anti-cellulite agents, moisturizers, sunscreens, skin lightening agents and skin tanning agents.

15. A hair care formulation containing a composition according to claim 1.

16. A “wash-off” formulations containing a composition according to claim 1.

17. A cosmetic or dermatologic formulation containing a composition according to claim 1 for the treatment of dry skin.

18. A cosmetic or dermatologic formulation containing a composition according to claim 1 for the treatment of aged skin.

19. A cosmetic or dermatologic formulation containing a composition according to claim 1 for improving the firmness of the skin.

20. The liquid and pumpable skin treatment composition according to claim 1 further including at least one of
D) 0.1 to 10% by weight consistency enhancers; and
E) auxiliaries and additives, with the proviso that A) to E) must adds up to 100% by weight.
